(19) United States
(12) Patent Application Publication
(10) Pub. No.: US 2024/0325024 A1
(43) Pub. Date: Oct. 3, 2024

(54) ANTI-MIGRATION DOUBLE WALLED FLANGE STENT

(71) Applicant: BOSTON SCIENTIFIC SCIMED, INC., Maple Grove, MN (US)

(72) Inventors: David Collins, Galway (IE); Darren Gerard Curran, Galway (IE); John O'Driscoll, Galway (IE); Fionn Stapleton, Galway (IE); Ryan Desmond Lynch, Roscommon (IE); Jack Mitchell, Galway (IE)

(73) Assignee: BOSTON SCIENTIFIC SCIMED, INC., Maple Grove, MN (US)

(21) Appl. No.: 18/618,770

(22) Filed: Mar. 27, 2024

Related U.S. Application Data
(60) Provisional application No. 63/492,665, filed on Mar. 28, 2023.

(51) Int. Cl. A61B 17/11; A61F 5/00

(52) U.S. Cl. CPC A61B 17/1114; A61F 5/0079; A61B 2017/1139

(57) ABSTRACT

A stent including a tubular body having first and second opposing open ends and a lumen extending therebetween. The stent including a first anchor member disposed adjacent the first open end and a second anchor member disposed adjacent the second open end, the first and second anchor members spaced apart by a saddle region of the tubular body, the first and second anchor members each extending radially outward from the tubular body, the first and second anchor members each have an outer diameter larger than an outer diameter of the saddle region disposed between the first and second anchor members. Each of the first and second anchor members include an outer wall, and an inner wall spaced apart from the outer wall, and the tubular body is defined by only a single wall.

### CROSS-REFERENCE TO RELATED APPLICATIONS

This application claims the benefit of U.S. Provisional Patent Application Ser. No. 63/492,665, filed Mar. 28, 2023, which is incorporated herein by reference.

### TECHNICAL FIELD

The present invention relates to devices, methods and systems for implanting stents. More particularly, the present invention relates to implantable stents for forming an anastomosis.

### BACKGROUND

An intraluminal prosthesis is a medical device used in the treatment of bodily lumens. One type of intraluminal prosthesis used in the repair and/or treatment of diseases in various body vessels is a stent. A stent is a generally longitudinal tubular device formed of biocompatible material which is useful to open and support various lumens in the body and/or form a conduit between body lumens. For example, stents may be used in the vascular system, urogenital tract, gastrointestinal tract, esophageal tract, tracheal/bronchial tubes and bile duct, as well as in a variety of other applications in the body.

Lumen apposing metal stents (LAMS) are used to drain pseudocysts, pancreatic fluid collections and to provide direct biliary and gallbladder drainage. LAMS may be used in other locations where a conduit crossing the walls between two duct structures is desired. Accordingly, there is an ongoing need to provide alternative stent structures that provide such conduits.

### SUMMARY

This disclosure provides design, material, manufacturing method, and use alternatives for medical devices. An example stent includes a tubular body formed of one or more interwoven wires, the tubular body having first and second opposing open ends and a lumen extending therebetween, the tubular body defining a longitudinal axis and a length extending between the first and second open ends, and a first anchor member disposed adjacent the first open end and a second anchor member disposed adjacent the second open end, the first and second anchor members spaced apart by a saddle region of the tubular body, the first and second anchor members each extending radially outward from the saddle region such that the first and second anchor members each have an outer diameter larger than an outer diameter of the saddle region disposed between the first and second anchor members, wherein each of the first and second anchor members include an outer wall and an inner wall spaced apart from the outer wall, wherein the tubular body is defined by only a single wall.

Alternatively or additionally to the embodiment above, the outer walls of each of the first and second anchor members are interwoven with the tubular body.

Alternatively or additionally to any of the embodiments above, the inner wall defines an inner space within each of the first and second anchor members that is continuous with the lumen of the tubular body.

Alternatively or additionally to any of the embodiments above, the outer wall is attached to the inner wall only at a base of each of the first and second anchor members, wherein the base is adjacent the tubular body.

Alternatively or additionally to any of the embodiments above, the first and second anchor members are formed as separate elements attached to the tubular body such that the tubular body extends across a base of each of the first and second anchor members and the inner wall defines an inner space separated from the lumen by the interwoven wires forming the tubular body.

Alternatively or additionally to any of the embodiments above, the tubular body includes a first end region extending between the first open end and the first anchor member and a second end region extending between the second open end and the second anchor member, wherein the outer diameter of the first and second anchor members is larger than an outer diameter of the first and second end regions.

Alternatively or additionally to any of the embodiments above, the outer diameters of the saddle region, the first end region, and the second end region are all substantially the same.

Alternatively or additionally to any of the embodiments above, the first and second anchor members are less flexible than the tubular body.

Alternatively or additionally to any of the embodiments above, the stent further comprises a covering extending over the tubular body, wherein an entirety of each of the first and second anchor members are devoid of the covering.

Alternatively or additionally to any of the embodiments above, the stent further comprises a covering extending over an entirety of the tubular body and the inner wall of each of the first and second anchor members.

Alternatively or additionally to any of the embodiments above, the outer wall of each of the first and second anchor members is devoid of the covering.

Alternatively or additionally to any of the embodiments above, the first and second anchor members extend perpendicular to the longitudinal axis.

Another example stent includes a tubular body formed of one or more interwoven wires, the tubular body having first and second opposing open ends and a lumen extending therebetween, the tubular body defining a longitudinal axis, and first and second longitudinally spaced apart anchor members each having a base adjacent the tubular body and extending radially outward to a free end, the first and second anchor members each having an outer diameter larger than an outer diameter of the tubular body disposed between the first and second anchor members, each of the first and second anchor members define a double-walled flange including a generally U-shaped outer wall and a generally U-shaped inner wall spaced apart from the outer wall, wherein the inner wall is fixed to the outer wall only at the base such that a radially outermost free end of the inner wall floats freely within the outer wall.

Alternatively or additionally to any of the embodiments above, the outer walls and/or the inner walls of each of the first and second anchor members are interwoven with the tubular body.

Alternatively or additionally to any of the embodiments above, the inner wall defines an inner space within each of the first and second anchor members that is continuous with the lumen of the tubular body.

Alternatively or additionally to any of the embodiments above, the first and second anchor members are formed as separate elements attached to the tubular body such that the tubular body extends across the base of each of the first and second anchor members and the inner wall defines an inner space separated from the lumen by the interwoven wires forming the tubular body.

Alternatively or additionally to any of the embodiments above, the tubular body includes a first end region extending between the first open end and the first anchor member and a second end region extending between the second open end and the second anchor member, wherein the outer diameter of the first and second anchor members is larger than an outer diameter of the first and second end regions, wherein the outer diameters of the tubular body between the first and second anchor members, the first end region, and the second end region are all substantially the same.

Alternatively or additionally to any of the embodiments above, the stent further comprises a covering extending over the tubular body, wherein the inner wall and the outer wall of each of the first and second anchor members are devoid of the covering.

Alternatively or additionally to any of the embodiments above, the stent further comprises a covering extending over an entirety of the tubular body and the inner wall of each of the first and second anchor members, wherein the outer wall of each of the first and second anchor members is devoid of the covering.

An example method of forming an anastomosis between first and second spaced apart body organs includes implanting a stent through a first tissue wall of a first body organ and a second tissue wall of a second body organ with a first open end of the stent disposed within the first body organ and a second open end of the stent disposed within the second body organ, the stent including a tubular body formed of one or more interwoven wires, the tubular body defining a lumen extending between the first and second open ends, the stent including first and second longitudinally spaced apart anchor members extending radially outward from the tubular body, wherein the first and second anchor members each have an outer diameter larger than an outer diameter of a saddle region extending between the first and second anchor members, wherein each of the first and second anchor members include an outer wall and an inner wall spaced apart from the outer wall, wherein the tubular body is defined by only a single wall, wherein the stent is implanted with the first tissue wall of the first body organ and the second tissue wall of the second body organ disposed within the saddle region, and with the first anchor member disposed within the first body organ and the second anchor member disposed within the second body organ, and draining fluid and particulates from the first body organ through the lumen of the stent into the second body organ.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

In the drawings, which are not necessarily drawn to scale, like numerals may describe similar components in different views. The drawings illustrate generally, by way of example, but not by way of limitation, various embodiments discussed in the present document.

FIGS. 1A-1C illustrate a gastrojejunostomy procedure;

FIG. 2 is a side cross-sectional view of a prior art stent;

FIG. 3 is a side cross-sectional view of an example tubular stent in accordance with an embodiment of the disclosure implanted across first and second body organs;

FIG. 4A is a side cross-sectional view of the tubular stent of FIG. 3 with the individual stent wires not shown for clarity;

FIG. 4B shows an enlarged view of region 4B of the tubular stent shown in FIG. 4A;

FIG. 5 is a side cross-sectional view of another example tubular stent in accordance with another embodiment of the disclosure; and

FIG. 6 is a side cross-sectional view of a further example tubular stent in accordance with a further embodiment of the disclosure.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

Lumen apposing metal stents (LAMS) may be used for creating and maintaining an alternative path of flow between two body lumens. For example, LAMS may be used for a gastro-jejunum or similar anastomosis that bypasses the proximal gut. LAMS are designed to cross organ tissue walls and act as a conduit between two body organs. LAMS may be used in a metabolic endoscopy or Natural Orifice Transluminal Endoscopic Surgery (NOTES) procedure to form an anastomosis (gastrojejunostomy) between the stomach and jejunum, which facilitates flow of food particulates and liquid from the stomach to the lower gastrointestinal tract, bypassing the pylorus and duodenum. This procedure may be used as a less invasive treatment for obesity and type 2 diabetes which are typically treated with Roux-en Y surgical bypass.

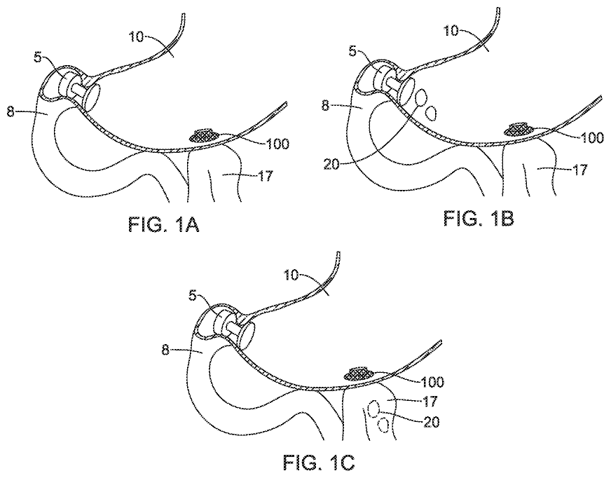

A gastrojejunostomy procedure is illustrated in FIGS. 1A-IC. In FIG. 1A, a plug 5 has been placed between the stomach 10 and the duodenum 8 to block the pylorus. An anastomosis is formed between another region of the stomach 10 and the jejunum 17 using a LAMS device 100. As shown in FIG. 1B, food particles 20 are blocked from passing from the stomach 10 through the pylorus into the duodenum 8 and upper 1.5 meters of the small intestine where most of the fats and nutrients are digested. Instead, as shown in FIG. 1C, food particles 20 pass through the LAMS device 100 from the stomach 10 directly into the jejunum 17 and lower region of the small intestine. The LAMS device 100 may be placed such a desired region of the small intestine is bypassed. For example, the first 0.5 to 2.0 meters of the small intestine are bypassed. In one example, the first 1.5 meters of the small intestine may be bypassed.

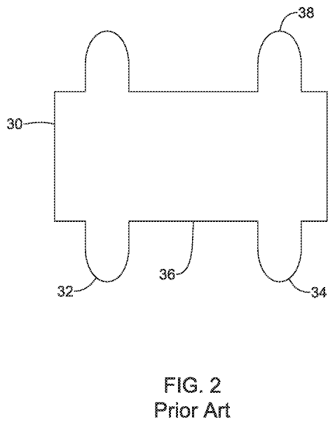

Based on initial preclinical studies, a 33% migration rate has been observed on an animal model using the prior art stent 30 shown in FIG. 2. FIG. 2 is a side cross-sectional view of the stent, with the individual wires forming the stent not shown to better illustrate the structure of the anchor members. This stent design includes a fully silicone coated nitinol prior art stent 30 with a first anchor member 32 and second anchor member 34 positioned on either side of a saddle region 36 which promotes the formation of an anastomosis. The first anchor member 32 and the second anchor member 34 are each formed with a single wall 38. The single wall 38 defining each flange is generally formed from the wires forming the remainder of the stent. The silicone covering on the stent is designed to prevent leakage of the food particles and liquid during anastomosis formation. On some occasions when the anastomosis is formed, migration of the stent has been observed on the animal model. Indications for in-dwell duration for the stent in the metabolic space between the stomach and small intestine is moving from a maximum of 60 days to upwards of 6-12 months. This indicates a growing need for LAMS with more robust anti-migration design features.

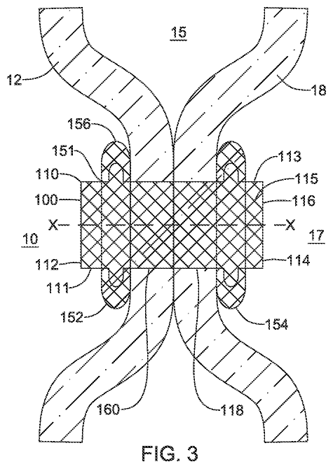

A stent 100 with a first anchor member 152 and a second anchor member 154 configured to maintain tissue apposition, and a body or saddle region 118 that applies a radial force to allow the formation of the anastomosis while also having the material characteristics that allow the opposition tissue to flex and move independently, is shown in FIG. 3. The stent 100 is shown in position providing a gastrojejunostomy anastomosis, with the stent extending between the stomach 10 and the jejunum 17 and spanning the inter-vessel cavity 15 therebetween, with the tissue wall 12 of the stomach 10 and the tissue wall 18 of the jejunum 17 sandwiched between the first anchor member 152 and the second anchor member 154, in the saddle region 118. It is noted that the stent 100 may be used to bridge between the walls of any anatomical structures. The stent 100 may have a tubular body 110 with a first open end 112 and a second open end 114, and a lumen extending therethrough. The tubular body 110 may be formed from one or more, or a plurality of interwoven wires 115 that may be woven, braided, wound, knitted, and combinations thereof, to form the tubular body 110. The stent 100 may include multiple wires 115 of a metal material, such as nitinol or nitinol-containing material, or another nickel-titanium alloy, for example. In some instances, the wires 115 may have a diameter of about 0.011 inches, for example. The number of wires 115 and the diameters of the wires 115, which may be the same or different, depicted in FIG. 3 are not limiting, and other numbers of wires 115 and other wire diameters may suitably be used. Desirably, an even number of wires 115 may be used, for example, from about 10 to about 36 wires 115.

Desirably, the wires 115 are made from any suitable implantable material, including without limitation nitinol, stainless steel, cobalt-based alloy such as Elgiloy®, platinum, gold, titanium, tantalum, niobium, polymeric materials and combinations thereof. Useful and nonlimiting examples of polymeric stent materials include poly(L-lactide) (PLLA), poly(D,L-lactide) (PLA), poly(glycolide) (PGA), poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-co-trimethylene carbonate) (PGA/PTMC), polydioxanone (PDS), Polycaprolactone (PCL), polyhydroxybutyrate (PHBT), poly(phosphazene) poly(D,L-lactide-co-caprolactone) PLA/PCL), poly(glycolide-co-caprolactone) (PGA/PCL), poly(phosphate ester) and the like. Wires made from polymeric materials may also include radiopaque materials, such as metallic-based powders, particulates or pastes which may be incorporated into the polymeric material. For example the radiopaque material may be blended with the polymer composition from which the polymeric wire is formed, and subsequently fashioned into the stent 100 as described herein. Alternatively, the radiopaque material may be applied to the surface of the metal or polymer wire 115 of the stent 100. In either instance, various radiopaque materials and their salts and derivatives may be used including, without limitation, bismuth, barium and its salts such as barium sulphate, tantalum, tungsten, gold, platinum and titanium, to name a few. Additional useful radiopaque materials may be found in U.S. Pat. No. 6,626,936, the contents of which are incorporated herein by reference. Metallic complexes useful as radiopaque materials are also contemplated. The stent may be selectively made radiopaque at desired areas along the wire or may be fully radiopaque.

In some instances, the wires 115 may have a composite construction having an inner core of tantalum, gold, platinum, tungsten, iridium or combination thereof and an outer member or layer of nitinol to provide a composite wire for improved radiopacity or visibility. In one example, the inner core may be platinum and the outer layer may be nitinol. The inner core of platinum may represent about at least 10% of the wire 115 based on the overall cross-sectional percentage. Moreover, nitinol that has not been treated for shape memory such as by heating, shaping and cooling the nitinol at its martensitic and austenitic phases, is also useful as the outer layer. Further details of such composite wires may be found in U.S. Pat. No. 7,101,392, the contents of which is incorporated herein by reference. The wires 115 may be made from nitinol, or a composite wire having a central core of platinum and an outer layer of nitinol. Further, the filling weld material, if required by welding processes such as MIG, may also be made from nitinol, stainless steel, cobalt-based alloy such as Elgiloy, platinum, gold, titanium, tantalum, niobium, and combinations thereof.

The tubular body 110 defines a lumen 116 extending longitudinally between the first and second open ends 112, 114. The first anchor member 152 may be disposed adjacent the first open end 112 and the second anchor member 154 may be disposed adjacent the second open end 114, where the first and second anchor members 152, 154 are longitudinally spaced apart by the saddle region 118. The first and second anchor members 152, 154, may both be self-expandable from a compressed delivery configuration in which the anchor members 152, 154 extend substantially parallel to the longitudinal axis x-x. In another example, the first and second anchor members 152, 154 are balloon-expandable. Each of the first and second anchor members 152, 154 may extend from a base 151 adjacent the tubular body 110 radially outward to a free end 156, forming flanges. In the example shown in FIG. 3, the first and second anchor members 152, 154 are symmetrical and the same size, and extend continuously and circumferentially around the tubular body 110. In other embodiments, the first anchor member 152 and the second anchor member 154 may be different sizes, such as having different longitudinal widths or different radial lengths. The first and second anchor members 152, may extend radially outward from the tubular body 110, substantially perpendicular to the longitudinal axis x-x of the stent 100. The first and second anchor members 152, 154 may each have an outer diameter larger than the outer diameter of the saddle region 118 disposed between the first and second anchor members 152, 154. The first anchor member 152 may have an outer diameter larger than the outer diameter of a first end region 111 of the tubular body 110 extending between the first open end 112 and the first anchor member 152, and the second anchor member 154 may have an outer diameter larger than the outer diameter of a second end region 113 of the tubular body 110 extending between the second open end 114 and the second anchor member 154. The outer diameter of both the first and second anchor members 152, 154 may be larger than the outer diameters of both the first and second end regions 111, 113. In some examples, the saddle region 118, the first end region 111, and the second end region 113 may all have substantially the same outer diameter.

The first and/or second anchor members 152, 154 may be formed of portions of the wires 115 forming the tubular body 110. In other embodiments, the first and/or second anchor members 152, 154 may be formed separately from the tubular body 110 and attached to the outer surface of the tubular body, such as by welding or with adhesive. The first and/or second anchor members 152, 154 may be less flexible than the tubular body 110. When the first and/or second anchor members 152, 154 are formed from the same wires 115 as the tubular body 110, the weaving, braiding, or knitting pattern of the first and/or second anchor members 152, 154 may be different than (e.g., tighter than) that of the tubular body 110 to make the anchor members 152, 154 less flexible than the tubular body 110. Alternatively, when the first and/or second anchor members 152, 154 are formed separately, they may be formed from less flexible wires or may include more wires in a pattern that makes them less flexible than the tubular body 110. Regardless of whether the first and/or second anchor members 152, 154 are formed from the same wires as the tubular body 110 or are formed separately and attached to the tubular body 110, the base 151 of the first and second anchor members 152, 154 may be stationary relative to the saddle region 118 to retain the tissue walls 18, 12 in apposition therebetween.

The stent 100 may include a covering 160 extending along the tubular body 110, such as along an interior and/or exterior of the tubular body 110. In some examples, the covering 160 extends over the entire outer surface of the stent, 100 including the tubular body 110 and first and second anchor members 152, 154. In other examples, the covering 160 extends along the entire inner surface of the stent, 100 including the tubular body 110 and first and second anchor members 152, 154. The covering 160 may fully cover the entire length of the stent 100, forming a fully covered stent in which all of the interstices defined by the interwoven wire(s) (e.g., interstices in the braided or woven pattern) are covered with the covering 160, thereby preventing tissue in-growth into the lumen 116 of the stent 100 and fluid leakage from the lumen 116 of the stent 100 into the inter-vessel cavity 15. In other examples, the covering 160 may cover only a portion of the length of the stent 100, forming a partially covered stent in which a portion of the interstices defined by the interwoven wire(s) (e.g., interstices in the braided or woven pattern) remain uncovered, allowing tissue in-growth in uncovered portions of the stent 100. For example, the covering 160 may be disposed only over the saddle region 118 of the stent 100 that will be disposed in the inter-vessel cavity 15. The covering 160 may be disposed on the inner or outer surface of the saddle region 118. In some examples, the covering 160 may be a solid walled sleeve, such as a silicone sleeve. An entirety of each of the first and/or second anchor members 152, 154 and the first and/or second end regions 111, 113 being devoid of the covering 160. This will allow for tissue in-growth into the first and/or second anchor members 152, 154, which may aid in preventing migration of the stent 100. The covering 160 may be expandable and contractible with any expansion and contraction of the stent 100. The covering 160 on the outer surface of the stent may aid in gripping the tissue walls 18, 12 and may protect the tissue walls 18, 12 from any damage that may result from wires forming the stent 100 impinging on the tissue walls 18, 12.

In some examples, the stent 100 may be spray coated or dip coated with the polymer covering 160, such as by spraying the stent 100 with a solution of silicone or other polymer, or dipping the stent 100 into a solution of silicone or other polymer to form the covering 160. In other examples, a polymer sheet or tube may be placed around or within the stent 100 to form the covering 160. The covering 160 may be disposed on external or internal surfaces of the stent 100, or on both the internal and external surfaces of the stent 100, thereby embedding the stent 100 in the polymeric material. The covering 160 may be a polymer covering, such as a polytetrafluoroethylene (PTFE) or silicone covering, however other coverings, particularly elastomeric polymers, may be used. Non-limiting examples of useful polymeric materials include polyesters, polypropylenes, polyethylenes, polyurethanes, polynaphthalenes, polytetrafluoroethylenes, expanded polytetrafluoroethylene, silicone, and combinations and copolymers thereof.

The stent 100 is configured to continuously flex and move as the tissue walls 12, 18 move, which may reduce the forces acting on an individual anchor member, which may reduce migration rates of the stent 100. In addition to metabolic procedures, the stent design may be used in other applications and body regions where migration is a concern.

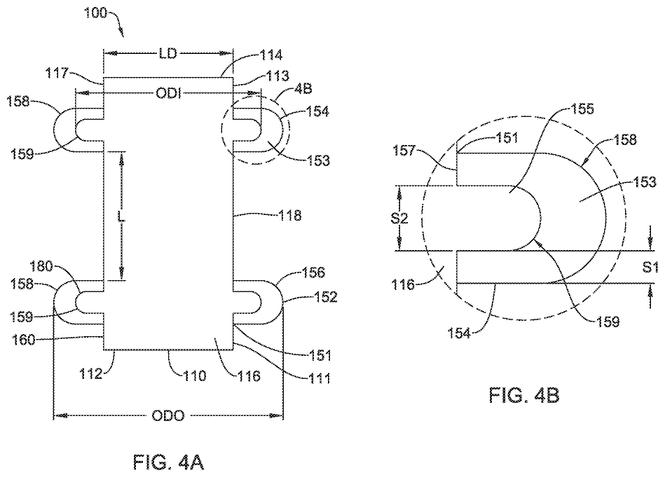

FIG. 4A shows a longitudinal cross-sectional view of the stent 100 of FIG. 3 with the individual wires forming the stent not shown to allow for a clear view of the double wall structure of the first anchor member 152 and second anchor member 154 on either side of the saddle region 118. FIG. 4B shows a close-up view of a portion of the second anchor member 154 of FIG. 4A. The double wall structure may include an outer wall 158 and an inner wall 159 spaced apart from the outer wall 158 by a first inner space 153. In some instances, the outer wall 158 may be a generally U-shaped wall extending circumferentially around the stent 100 with first and second ends of the outer wall 158 located at the base 151 of the corresponding anchor member 152, 154 and an arcuate portion of the wall positioned at a radially outward most extent or free end of the outer wall 158. Thus, the outer wall 158 may extend outward from the tubular body 110 at the base 151 for a distance (e.g., radial extent of the outer wall 158) and then turn back toward the tubular body 110 and extend inward to the tubular body 110. Similarly, the inner wall 159 may be a generally U-shaped wall extending circumferentially around the stent 100 with first and second ends of the inner wall 159 located at the base 151 of the corresponding anchor member 152, 154 and an arcuate portion of the wall positioned at a radially outward most extent or free end of the inner wall 159. Thus, the inner wall 159 may extend outward from the tubular body 110 at the base 151 for a distance (e.g., radial extent of the inner wall 159) and then turn back toward the tubular body 110 and extend inward to the tubular body 110. The outermost extent or free end of the outer wall 158 may be greater than the outermost extent or free end of the inner wall 159, providing a radially extending gap therebetween. In some instances, the inner space 153 may be a generally U-shaped inner space extending circumferentially around the stent 100 spacing the exterior side of the inner wall 159 apart from the interior side of the outer wall 158.

The inner wall 159 may be interwoven with the tubular body 110 such that the tubular body 110 and the inner walls 159 of each of the first and second anchor members 152, 154 is a single monolithic structure. In other words, interwoven wires forming the tubular body 110 may extend continuously from the tubular body 110 to form the inner wall 159 of the first anchor member 152 and/or the second anchor member 154.

Alternatively or additionally, the outer wall 158 may be interwoven with the tubular body 110 such that an entirety of the outer surface of the stent 100, including the tubular body 110 and outer walls 158 of each of the first and second anchor members 152, 154 is a single monolithic structure. In other words, interwoven wires forming the tubular body 110 may extend continuously from the tubular body 110 to form the outer wall 158 of the first anchor member 152 and/or the second anchor member 154.

In some instances, a plurality of interwoven wires 115 may extend continuously from the first open end 112 to the second open end 114 such that the interwoven wires 115 extend continuously through the first end region 111, the saddle region 118 and the second end region 113. The interwoven wires 115 may be divided into a first subset of wires and a second subset of wires through the first anchor member 152, with the first subset of wires forming only the outer wall 158 of the first anchor member 152 and the second subset of wires forming only the inner wall 159 of the first anchor member 152. In other words, a first subset of the plurality of interwoven wires 115 may extend from the first end region 111 to form the outer wall 158 of the first anchor member 152 and then back to form the saddle region 118, while a second subset of the plurality of interwoven wires 115 may extend from the first end region 111 to form the inner wall 159 of the first anchor member 152 and then back to form the saddle region 118. The first subset of wires, forming the outer wall 158 may be exclusive of the second subset of wires forming the inner wall 159, thus wires utilized in forming the outer wall 158 of the first anchor member 152 are not utilized in forming the inner wall 159 of the first anchor member 152.

Likewise, the interwoven wires 115 may be divided into a first subset of wires and a second subset of wires through the second anchor member 154, with the first subset of wires forming only the outer wall 158 of the second anchor member 154 and the second subset of wires forming only the inner wall 159 of the second anchor member 154. In other words, a first subset of the plurality of interwoven wires 115 may extend from the second end region 113 to form the outer wall 158 of the second anchor member 154 and then back to form the saddle region 118, while a second subset of the plurality of interwoven wires 115 may extend from the second end region 113 to form the inner wall 159 of the second anchor member 154 and then back to form the saddle region 118. The first subset of wires, forming the outer wall 158 may be exclusive of the second subset of wires forming the inner wall 159, thus wires utilized in forming the outer wall 158 of the second anchor member 154 are not utilized in forming the inner wall 159 of the second anchor member 154.

The first subset of wires forming the outer wall 158 of the first anchor member 152 may be the same as, or different than the first subset of wires forming the outer wall 158 of the second anchor member 154. Likewise, second subset of wires forming the inner wall 159 of the first anchor member 152 may be the same as, or different than the second subset of wire forming the inner wall 159 of the second anchor member 154.

Thus, each of the inner wall 158 and the outer wall 158 of the first and second anchor members 152, 154 may be formed of only a portion of the interwoven wires 115 forming the saddle region 118, the first end region 111, and/or the second end region 113 of the stent 100.

In other instances the inner wall 159 and/or the outer wall 158 may be formed separately from and subsequently attached to the tubular wall 110. For example, the inner wall 159 may be formed integrally with the plurality of interwoven wires forming the tubular body 110, and then the outer wall 158 may be subsequently attached around the inner wall 159.

The inner wall 159 may be fixed to the outer wall 158 only at the base 151 of the first and second anchor members 152, 154. The base 151 is adjacent the tubular body 110. The remainder of the inner wall 159 including its free end 180 floats freely within the outer wall 158. The radially outward portions of the outer wall 158 and the inner wall 159 move independently of each other. For instance, the radially outward portion of the outer wall 158 (e.g., the arcuately curved radially outward most portion of the outer wall 158) of one of the first anchor member 152 and/or the second anchor member 154 may deflect toward the first open end 112 or the second open end 114 of the stent 100 without deflecting the corresponding inner wall 159 of the same anchor member 152, 154.

In some examples, the inner wall 159 may be connected to the outer wall 158 by a connection region 157 of the tubular body 110, as shown in FIG. 4B. The connection region 157 may extend substantially parallel to the longitudinal axis. The inner wall 159 defines a second inner space 155 within an interior of the inner wall 159. In some examples, the second inner space 155 is continuous with the lumen 116 of the tubular body 110.

When a covering 160 is present on the tubular body 110, the covering may extend over both the outer wall 158 and the inner wall 159 of each of the first and second anchor members 152, 154. In other embodiments, the outer wall 158 of each of the first and second anchor members 152, 154 may be devoid of any covering, while the inner wall 159 of each of the first and second anchor members 152, 154 may be covered by the covering 160. The inner walls 159 may be covered with a polymer or silicone sleeve. Thus, in some instances the covering 160 may extend the entire length of the stent 100, covering the first end region 111, the inner wall 159 of the first anchor member 152, the saddle region 118, the inner wall 159 of the second anchor member 154, and the second end region 113, while the outer walls 158 of each of the first and second anchor members 152, 154 remains uncovered or devoid of the covering 160. This may allow for the outer walls 158 to provide tissue in-growth into interstices formed between interwoven wires forming the outer walls 158 to prevent migration, while the inner walls 159 may be covered to prevent tissue ingrowth into the lumen 116 and provide a fluid tight conduit through the lumen 116. In other examples, the outer walls 158 may be covered with the covering 160, and the inner walls 159 may be devoid of the covering.

The double wall structure of the first and second anchor members 152, 154 may be in contrast to the tubular body 110, which may be defined by only a single wall 117. In other examples, at least a portion of the tubular body 110 may be formed with a double wall. For example, the saddle region 118 may have a double wall construction with a first wall formed of interwoven wires radially surrounding a second wall formed of interwoven wires. The first, or outer wall of the double-walled saddle region 118 may be formed of the same wires or different wires from the second, or inner wall of the double-walled saddle region 118.

The outer walls 158 of each of the first and second anchor members 152, 154 may be interwoven with the tubular body 110. For example, the interwoven wires 115 forming the tubular body 110 may be extended radially outward to form the outer walls 158 of each of the first and second anchor members 152, 154, such that the entire tubular body 110 and the outer walls 158 of the first and second anchor members 152, 154 are formed continuously from the same wires 115.

Additionally or alternatively, the inner walls 159 of each of the first and second anchor members 152, 154 may be interwoven with the tubular body 110. For example, the interwoven wires 115 forming the tubular body 110 may be extended radially outward to form the inner walls 159 of each of the first and second anchor members 152, 154, such that the entire tubular body 110 and the inner walls 159 of the first and second anchor members 152, 154 are formed continuously from the same wires 115. In some examples, the inner wall 159 defines a second inner space 155 within each of the first and second anchor members 152, 154 that is continuous with the lumen 116 of the tubular body.

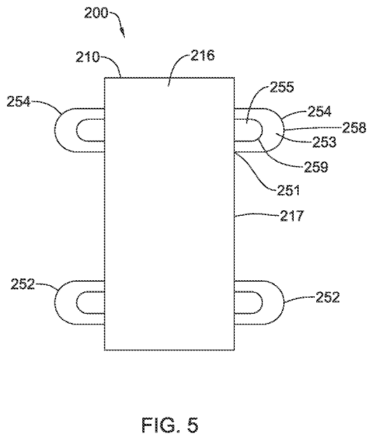

In another example, the tubular body 210 of a stent 200 may include first and second anchor members 252, 254 formed as separate elements that are attached to the tubular body 210. See FIG. 5. The first and second anchor members 252, 254 may be formed from one or more wires in a similar manner as the tubular body 210 and attached in any suitable manner, including with welding or adhesive. The first and second anchor members 252, 254 may each include an outer wall 258 and an inner wall 259 separately attached to the tubular body 210 at spaced apart locations to define the first inner space 253 between the outer wall 258 and inner wall 259. The tubular body 210 may be formed with a single wall 217, and the single wall 217 may extend across the base 251 of each of the first and second anchor members 252, 254 such that the inner wall 259 of the first and second anchor members 252, 254 defines a second inner space 255 separated from the lumen 216 of the tubular body 210 by the interwoven wires forming the single wall 217 of the tubular body 210.

The double wall structure allows the stent to move and flex within the anastomosis without failing to maintain position. The inner wall 159 provides an extra layer or wall of wire within the outer wall 158 that gives longitudinal strength and increased pullout forces that will prevent migration of the stent 100. The extra wall of wire defined by the inner wall 159 may provide radial force to allow the formation of the anastomosis and maintain the lumen 116 in an open configuration to allow the passage of fluid and/or food. The outer and inner walls 158, 159 may move independently of each other due to them being joined to one another at only the base 151 of the first and second anchor members 152, 154, or being joined separately only to the wall 217 of the tubular body 210. This independent movement may allow the outer wall 158 of the second anchor member 154 to grip onto the gastric wall and the outer wall 158 of the first anchor member 152 to grip onto the jejunum wall, and if potential movement was to occur the inner wall 159 of each flange would provide additional strength to resist such movement.

The stent 100 may be sized to allow a desired rate of fluid flow therethrough. FIGS. 4A and 4B show the locations of the following measurements. In some examples, the stent may have a lumen 116 diameter (LD) of between 15 mm and 30 mm. The longitudinal length (L) of the saddle region 118 may be between 5 mm and 15 mm. The outer diameter of the inner wall 159 (ODI) of the first and second anchor members 152, 154 may be between 20 mm and 35 mm, and the outer diameter of the outer wall 158 (ODO) of the first and second anchor members 152, 154 may be between 25 mm and 40 mm. In some examples, the spacing (S1) between the inner wall 159 and the outer wall 158, measured longitudinally, may be between 0.5 mm and 1.5 mm. In some examples, the spacing (S2) between opposing ends of the inner wall 159, measured longitudinally at the base 151, may be between 1.5 mm and 2.5 mm. In one example, the LD may be about 20 mm, the L may be about 10 mm, the ODI may be about 25 mm, the ODO may be about 30 mm, the S1 may be about 1 mm, and the S2 may be about 2 mm.

A method of forming an anastomosis between first and second spaced apart body organs involves implanting a stent 100 through a first tissue wall 12 of a first body organ 10 and a second tissue wall 18 of a second body organ 17 such that a first open end 112 of the stent 100 is disposed within the first body organ 10 and a second open end 114 of the stent 100 is disposed within the second body organ 17. The stent 100 may be delivered in a longitudinally extended and radially compressed configuration in which the first and second anchor members 152, 154 are compressed to substantially the same outer diameter as the first and second end regions 111, 113 and the saddle region 118. The stent 100 may be delivered through a catheter or endoscope. Upon removal from the catheter or endoscope, the stent 100 may self-expand to the configuration shown in FIG. 3. In other examples, the stent 100 may be expanded with a balloon or other mechanical device. The first and second anchor members 152, 154 may be self-expandable. The stent 100 including the tubular body 110 may be formed of one or more, or a plurality of interwoven wires 115, with the tubular body 220 defining a lumen 116 extending between the first and second open ends of the stent 100. The one or more, or plurality of interwoven wires 115 forming the tubular body 110 may also extend into and form the first and/or second longitudinally spaced apart anchor members 152, 154 extending radially outward from the tubular body 110. The first and second anchor members 152, 154 each have an outer diameter larger than an outer diameter of a saddle region 118 extending between the first and second anchor members 152, 154, and each of the first and second anchor members 152, 154 include an outer wall 158 and an inner wall 159 spaced apart from the outer wall 158. The tubular body 110 may be defined by only a single wall 117 formed of the interwoven wire(s). The stent 100 may be implanted with the first tissue wall 12 of the first body organ and the second tissue wall 18 of the second body organ disposed within the saddle region 118, and with the first anchor member 152 disposed within the first body organ 10 and the second anchor member 154 disposed within the second body organ 17. The method further includes draining fluid and particulates from the first body organ 10 through the lumen of the stent 100 into the second body organ 17.

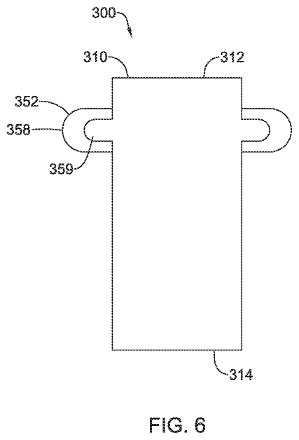

FIG. 6 is a side cross-sectional view of another example stent 300 including a tubular body 310 and a single anchor member 352 disposed adjacent the first open end 312. The anchor member 351 has a double wall construction including an inner wall 359 and an outer wall 358 as discussed above with regard to the first and second anchor members 152, 154 of stent 100. However, the stent 300 differs from stent 100 in that the stent 300 has only the single anchor member 352, and the second open end 314 is devoid of any anchor members adjacent thereto. The anchor member 352 may be formed similar to that described above. For instance, a first subset of wires forming the tubular body 310 may extend continuously to form the outer wall 358, while a second subset of wires forming the tubular body 310 may extend continuously to form the inner wall 359. In other instances, wires forming the tubular body 310 may extend continuously to form the inner wall 359, while the outer wall 358 may be formed separately from discrete interwoven wire and then attached to the tubular body 310. Other configurations are also contemplated. The stent 300 may be implanted across two organs with the anchor member 352 disposed in a first body organ and the remainder of the tubular body 310 and second open end 314 disposed in a second body organ. The tubular body 310 may have a length configured to maintain the second open end 314 disposed within the second body organ.

Various stent types and stent constructions may be employed for the stent 100, 200, 300. For example, the stent 100, 200, 300 may be a self-expanding stent or a balloon expandable stent. The stent 100, 200, 300 may be capable of radially contracting to a compressed or collapsed configuration for delivery, and then expandable to an expanded configuration during deployment in the body vessels. Thus, the stent 100, 200, 300 may be described as radially distensible or deformable. Self-expanding stents include those that have a spring-like action which causes the stent to radially expand, or stents which expand due to the memory properties of the stent material for a particular configuration at a certain temperature. The configuration of the stent may also be chosen from a host of geometries. For example, wire stents can be fastened into a continuous helical pattern, with or without a wave-like or zig-zag in the wire, to form a radially deformable stent. Individual rings or circular members can be linked together such as by struts, sutures, welding or interlacing or locking of the rings to form a tubular stent. In other embodiments, the stent 100, 200, 300 may be formed as a monolithic tubular member by etching or cutting a pattern of interconnected struts from a tube.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A stent comprising:
a tubular body formed of one or more interwoven wires, the tubular body having first and second opposing open ends and a lumen extending therebetween, the tubular body defining a longitudinal axis and a length extending between the first and second open ends; and
a first anchor member disposed adjacent the first open end and a second anchor member disposed adjacent the second open end, the first and second anchor members spaced apart by a saddle region of the tubular body, the first and second anchor members each extending radially outward from the saddle region such that the first and second anchor members each have an outer diameter larger than an outer diameter of the saddle region disposed between the first and second anchor members;
wherein each of the first and second anchor members include an outer wall and an inner wall spaced apart from the outer wall, wherein the tubular body is defined by only a single wall.

2. The stent of claim 1, wherein the outer walls of each of the first and second anchor members are interwoven with the tubular body.

3. The stent of claim 2, wherein the inner wall defines an inner space within each of the first and second anchor members that is continuous with the lumen of the tubular body.

4. The stent of claim 1, wherein the outer wall is attached to the inner wall only at a base of each of the first and second anchor members, wherein the base is adjacent the tubular body.

5. The stent of claim 1, wherein the first and second anchor members are formed as separate elements attached to the tubular body such that the tubular body extends across a base of each of the first and second anchor members and the inner wall defines an inner space separated from the lumen by the interwoven wires forming the tubular body.

6. The stent of claim 1, wherein the tubular body includes a first end region extending between the first open end and the first anchor member and a second end region extending between the second open end and the second anchor member, wherein the outer diameter of the first and second anchor members is larger than an outer diameter of the first and second end regions.

7. The stent of claim 6, wherein the outer diameters of the saddle region, the first end region, and the second end region are all substantially the same.

8. The stent of claim 1, wherein the first and second anchor members are less flexible than the tubular body.

9. The stent of claim 1, further comprising a covering extending over the tubular body, wherein an entirety of each of the first and second anchor members are devoid of the covering.

10. The stent of claim 1, further comprising a covering extending over an entirety of the tubular body and the inner wall of each of the first and second anchor members.

11. The stent of claim 10, wherein the outer wall of each of the first and second anchor members is devoid of the covering.

12. The stent of claim 1, wherein the first and second anchor members extend perpendicular to the longitudinal axis.

13. A stent comprising:
a tubular body formed of one or more interwoven wires, the tubular body having first and second opposing open ends and a lumen extending therebetween, the tubular body defining a longitudinal axis; and
first and second longitudinally spaced apart anchor members each having a base adjacent the tubular body and extending radially outward to a free end, the first and second anchor members each having an outer diameter larger than an outer diameter of the tubular body disposed between the first and second anchor members, each of the first and second anchor members define a double-walled flange including a generally U-shaped outer wall and a generally U-shaped inner wall spaced apart from the outer wall, wherein the inner wall is fixed to the outer wall only at the base such that a radially outermost free end of the inner wall floats freely within the outer wall.

14. The stent of claim 13, wherein the outer walls and/or the inner walls of each of the first and second anchor members are interwoven with the tubular body.

15. The stent of claim 14, wherein the inner wall defines an inner space within each of the first and second anchor members that is continuous with the lumen of the tubular body.

16. The stent of claim 13, wherein the first and second anchor members are formed as separate elements attached to the tubular body such that the tubular body extends across the base of each of the first and second anchor members and the inner wall defines an inner space separated from the lumen by the interwoven wires forming the tubular body.

17. The stent of claim 13, wherein the tubular body includes a first end region extending between the first open end and the first anchor member and a second end region extending between the second open end and the second anchor member, wherein the outer diameter of the first and second anchor members is larger than an outer diameter of the first and second end regions, wherein the outer diameters of the tubular body between the first and second anchor members, the first end region, and the second end region are all substantially the same.

18. The stent of claim 13, further comprising a covering extending over the tubular body, wherein the inner wall and the outer wall of each of the first and second anchor members are devoid of the covering.

19. The stent of claim 13, further comprising a covering extending over an entirety of the tubular body and the inner wall of each of the first and second anchor members, wherein the outer wall of each of the first and second anchor members is devoid of the covering.

20. A method of forming an anastomosis between first and second spaced apart body organs, comprising:
implanting a stent through a first tissue wall of a first body organ and a second tissue wall of a second body organ with a first open end of the stent disposed within the first body organ and a second open end of the stent disposed within the second body organ, the stent including
a tubular body formed of one or more interwoven wires, the tubular body defining a lumen extending between the first and second open ends, the stent including first and second longitudinally spaced apart anchor members extending radially outward from the tubular body,
wherein the first and second anchor members each have an outer diameter larger than an outer diameter of a saddle region extending between the first and second anchor members, wherein each of the first and second anchor members include an outer wall and an inner wall spaced apart from the outer wall, wherein the tubular body is defined by only a single wall;
wherein the stent is implanted with the first tissue wall of the first body organ and the second tissue wall of the second body organ disposed within the saddle region, and with the first anchor member disposed within the first body organ and the second anchor member disposed within the second body organ; and
draining fluid and particulates from the first body organ through the lumen of the stent into the second body organ.
